SEQUENCE LISTING (8 sequences)

SEQ ID NO: 1 - protein, 5 aa, Artificial Sequence
  Synthetic construct
DTYIH

SEQ ID NO: 2 - protein, 17 aa, Artificial Sequence
  Synthetic construct
RIYPTNGYTRYADSVKG

SEQ ID NO: 3 - protein, 11 aa, Artificial Sequence
  Synthetic construct
WGGDGFYAMDY

SEQ ID NO: 4 - protein, 11 aa, Artificial Sequence
  Synthetic construct
RASQDVNTAVA

SEQ ID NO: 5 - protein, 7 aa, Artificial Sequence
  Synthetic construct
SASFLYS

SEQ ID NO: 6 - protein, 9 aa, Artificial Sequence
  Synthetic construct
QQHYTTPPT

SEQ ID NO: 7 - protein, 450 aa, Artificial Sequence
  Synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 214 aa, Artificial Sequence
  Synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0339548 A1
(43) Pub. Date: Nov. 6, 2025

(54) METHOD FOR TREATING CANCER BY USING ANTIBODY-DRUG CONJUGATE AND USE

(71) Applicant: SICHUAN KELUN-BIOTECH BIOPHARMACEUTICAL CO., LTD., Chengdu, Sichuan (CN)

(72) Inventors: Yan QING, Chengdu, Sichuan (CN); Shuli YI, Chengdu, Sichuan (CN); Juanjuan YUAN, Chengdu, Sichuan (CN); Hong CHEN, Chengdu, Sichuan (CN); Haochuan ZHENG, Chengdu, Sichuan (CN); Bin FAN, Chengdu, Sichuan (CN); Xuenong OUYANG, Chengdu, Sichuan (CN); Junyou GE, Chengdu, Sichuan (CN); Jingyi WANG, Chengdu, Sichuan (CN)

(73) Assignee: SICHUAN KELUN-BIOTECH BIOPHARMACEUTICAL CO., LTD., Chengdu, Sichuan (CN)

(21) Appl. No.: 18/549,459

(22) PCT Filed: Apr. 28, 2022
(86) PCT No.: PCT/CN2022/089836

§ 371 Date: Sep. 7, 2023

(30) Foreign Application Priority Data
May 7, 2021 (CN) 202110494940.0
May 18, 2021 (CN) 202110538130.0

(51) Int. Cl. A61K 47/68; A61K 9/00; A61K 47/02; A61K 47/26; A61P 35/00; C07K 16/32

(52) U.S. Cl. CPC A61K 47/68031; A61K 9/0019; A61K 47/02; A61K 47/26; A61K 47/6855; A61P 35/00; C07K 16/32

(57) ABSTRACT

The present invention belongs to the technical field of biomedicine, and relates to a method for treating cancer by using an antibody-drug conjugate. In particular, the present invention relates to a use of an antibody drug conjugate represented by formula (I) and a pharmaceutically acceptable salt, a stereoisomer or a metabolite thereof, or a solvate of the foregoing in the manufacture of a medicament for preventing and/or treating a HER2-expressing, amplified or mutated cancer, wherein A is a group obtained by removing n amino groups from an antibody against HER2 or an active fragment or variant thereof; preferably, A is trastuzumab or a group obtained by removing n amino groups from Pertuzumab; n is selected from 1-10, such as 2-10 or 2-8; and preferably, n is an integer of 1, 2, 3, 4, 5, 6, 7 or 8.

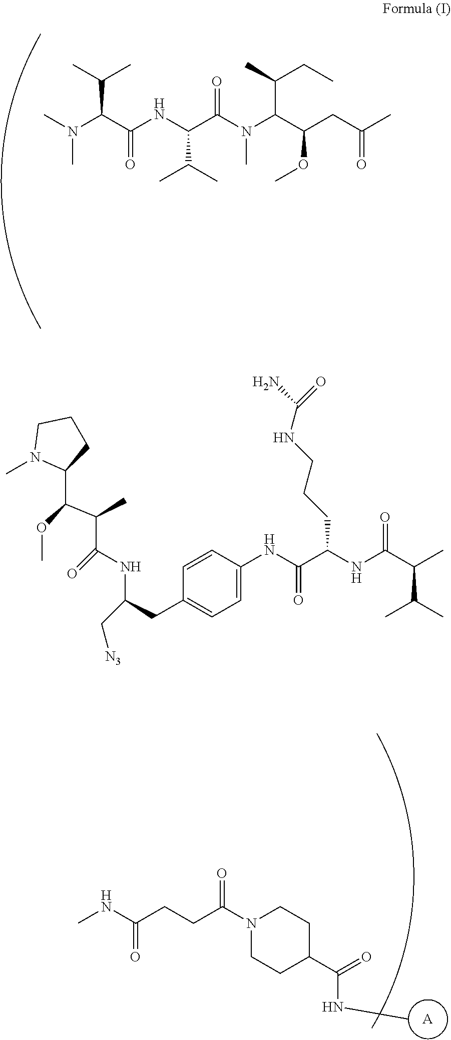

### CROSS REFERENCE TO RELATED APPLICATIONS

This application is a 35 U.S.C. § 371 National Phase Entry Application from PCT/CN2022/089836, filed Apr. 28, 2022, which claims the benefit of CN 202110494940.0 filed on May 7, 2021 and CN 202110538130.0 filed on May 18, 2021, the disclosures of which are incorporated herein in their entirety by reference.

### SEQUENCE LISTING

The instant application contains a Sequence Listing, which is hereby incorporated by reference in its entirety. Said Sequence Listing, created on Feb. 7, 2024, is named P2022TC2018_sequence listing-EN2 and is 7.3 kilobytes in size.

### TECHNICAL FIELD

The present invention belongs to the technical field of biomedicines. Specifically, the present invention relates to a method for treating cancers by using an antibody-drug conjugate, and to use of the antibody-drug conjugate in the manufacture of a medicament for treating cancers, in particular use of an anti-HER2 antibody-drug conjugate in the manufacture of a medicament for treating cancers.

### BACKGROUND ART

Malignant tumors are major public health problems in China and in the world. China is a country with high incidence of tumors (in which non-small cell lung cancer, gastric cancer, liver cancer, colorectal cancer and breast cancer are the tumors with top five incidences). Moreover, studies have found that a human epidermal growth factor receptor 2 (HER2) gene is expressed to different extents in many malignant tumors, especially in epithelium-derived tumors, such as breast cancer, lung cancer, ovarian cancer, gastric cancer, and colorectal cancer. In recent years, anti-tumor antibody drugs have been rapidly developed in terms of basic research and development, clinical application, etc. However, the existing antibody drugs have limited anti-tumor efficacy when used alone and are often used in combination with chemotherapy drugs clinically, and most patients who received an effective initial antibody therapy are prone to developing drug resistance. An antibody-drug conjugate (ADC) has unique advantages in the aspects of improving the therapeutic efficacy of an antibody drug, overcoming drug resistance and fully utilizing antibody targeting.

Among ADC drugs with HER2 as a major target, T-DM1 developed by Roche and DS-8201a developed by Daiichi-Sankyo are antibody-drug conjugates targeting HER2. All the indications for which T-DM1 is approved are HER2-positive breast cancer. The indications for which DS-8201a is approved are HER2-positive breast cancer, and DS-8201a is later approved by Pharmaceuticals and Medical Devices Agency (PMDA) for newly increased HER2-positive gastric cancer indications.

Thus, it is urgent and necessary to develop HER2 antibody-drug conjugates having good clinical efficacy on more types of cancers with HER2 expression, which will provide more drug choices for cancer patients.

### SUMMARY OF THE INVENTION

In a first aspect, the present invention provides a method of preventing and/or treating a cancer with HER2 expression, amplification, or mutation, comprising administering to a subject in need thereof a therapeutically effective amount of an antibody-drug conjugate of Formula (I), a pharmaceutically acceptable salt, a stereoisomer or a metabolite thereof, or a solvate of the foregoing,

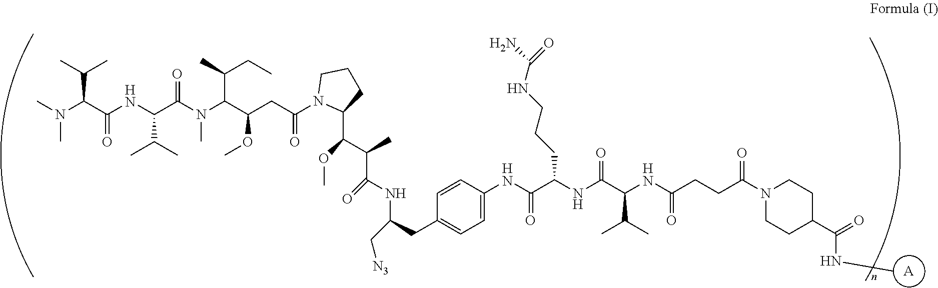

wherein
A is a moiety obtained by the removal of n amino groups from an anti-HER2 antibody or an active fragment or variant thereof, preferably a moiety obtained by the removal of n amino groups from trastuzumab or pertuzumab; and
n is an integer selected from a group consisting of 1-10, e.g., 2-10 or 2-8; preferably, n is an integer of 1, 2, 3, 4, 5, 6, 7 or 8.

In a second aspect, the present invention provides use of the antibody-drug conjugate of Formula (I), the pharmaceutically acceptable salt, the stereoisomer or the metabolite thereof, or the solvate of the foregoing in the manufacture of a medicament for the prevention and/or treatment of a cancer with HER2 expression, amplification, or mutation.

In a third aspect, the present invention provides the antibody-drug conjugate of Formula (I), the pharmaceutically acceptable salt, the stereoisomer or the metabolite thereof, or the solvate of the foregoing, for use of preventing and/or treating a cancer with HER2 expression, amplification, or mutation.

In a preferred embodiment, the antibody-drug conjugate of Formula (I) has a structure represented by the following Formula (I-1),

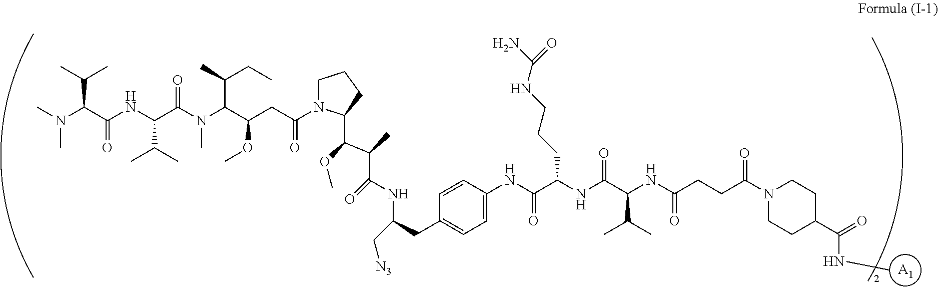

wherein A1 is a moiety obtained by the removal of 2 amino groups from trastuzumab.

In a preferred embodiment, the cancer with HER2 expression is a cancer with HER2 low-expression or a cancer with HER2 over-expression.

In a preferred embodiment, the cancer with HER2 low-expression is a cancer with an HER2 expression level of IHC (immunohistochemistry) 1+ or IHC 2+/FISH (fluorescence in situ hybridization) negative.

In a preferred embodiment, the cancer with HER2 over-expression is a cancer with an HER2 expression level of IHC 2+/FISH positive or IHC 3+.

In a preferred embodiment, the cancer with HER2 low-expression comprises salivary gland cancer, breast cancer, gastric cancer, gastroesophageal junction cancer, esophageal cancer, lung cancer, colorectal cancer, urothelial cancer, biliary tract cancer, head and neck cancer, ovarian cancer, endometrial cancer, cervical cancer, pancreatic cancer, liver cancer, etc.

In a preferred embodiment, the cancer with HER2 over-expression comprises breast cancer, gastric cancer, gastroesophageal junction cancer, esophageal cancer, lung cancer, colorectal cancer, urothelial cancer, salivary gland cancer, biliary tract cancer, head and neck cancer, ovarian cancer, endometrial cancer, cervical cancer, pancreatic cancer, liver cancer, gallbladder cancer, bladder cancer, etc.

In a preferred embodiment, the cancer with HER2 mutation comprises breast cancer, biliary tract cancer, ovarian cancer, lung cancer, colorectal cancer, salivary gland cancer, head and neck cancer, endometrial cancer, cervical cancer, liver cancer, upper gastrointestinal cancer, etc.

In a preferred embodiment, the cancer with HER2 amplification comprises breast cancer, gastroesophageal junction adenocarcinoma, ovarian cancer, colorectal cancer, lung cancer, gallbladder cancer, upper gastrointestinal cancer, etc.

In a preferred embodiment, the breast cancer comprises breast cancer with HER2 expression, or breast cancer which has been treated ineffectively with at least one anti-HER2 therapeutic regimen in the past, or the like.

In a preferred embodiment, the breast cancer with HER2 expression comprises unresectable breast cancer, and the breast cancer which has been treated ineffectively with at least one anti-HER2 therapeutic regimen in the past comprises HER2-positive unresectable breast cancer.

In a preferred embodiment, the unresectable breast cancer is unresectable locally advanced, recurrent or metastatic breast cancer, and the HER2-positive unresectable breast cancer is HER2-positive unresectable locally advanced, recurrent or metastatic breast cancer.

In a preferred embodiment, the breast cancer comprises unresectable locally advanced, recurrent or metastatic breast cancer with HER2 expression, or HER2-positive unresectable locally advanced, recurrent or metastatic breast cancer which has been treated ineffectively with at least one anti-HER2 therapeutic regimen in the past, or the like.

In a preferred embodiment, the at least one anti-HER2 therapeutic regimen comprises: (1) trastuzumab and/or pertuzumab combined chemotherapy; (2) tucatinib; (3) neratinib; (4) T-DM1; (5) pyrotinib combined chemotherapy; (6) lapatinib combined chemotherapy; and (7) tucatinib or pyrotinib or lapatinib+trastuzumab+chemotherapy, and the like. Therapeutic biological products such as trastuzumab can be replaced by biosimilars thereof.

In a preferred embodiment, the salivary gland cancer comprises one or more of parotid gland carcinoma, salivary duct carcinoma, adenoid cystic carcinoma, submaxillary gland carcinoma, sublingual gland carcinoma, lingual gland carcinoma, minor salivary gland carcinoma, labial gland carcinoma, and retromolar gland carcinoma.

In a preferred embodiment, the esophageal cancer is esophageal adenocarcinoma or esophageal squamous cell carcinoma.

In a preferred embodiment, the liver cancer is intrahepatic cholangiocarcinoma or hepatocellular carcinoma.

In a preferred embodiment, the upper gastrointestinal cancer is esophageal adenocarcinoma or biliary duct cancer.

In a preferred embodiment, the cancer with HER2 low-expression is salivary gland cancer, preferably parotid gland carcinoma with an HER2 expression level of IHC 2+ or salivary duct carcinoma with an HER2 expression level of IHC 1+.

In a preferred embodiment, the cancer with HER2 low-expression is head and neck cancer, preferably head and neck squamous cell carcinoma, more preferably head and neck squamous cell carcinoma with an HER2 expression level of IHC 2+.

In a preferred embodiment, the cancer with HER2 low-expression is breast cancer, preferably breast cancer with an HER2 expression level of IHC 1+.

In a preferred embodiment, the cancer with HER2 over-expression is selected from a group consisting of breast cancer, bladder cancer, colorectal cancer, gallbladder cancer, salivary gland cancer, and head and neck cancer, preferably breast cancer, bladder cancer, colorectal cancer, gallbladder cancer, parotid gland carcinoma or head and neck squamous cell carcinoma with an HER2 expression level of IHC 3+.

In a preferred embodiment, the cancer with HER2 expression may also be intrahepatic cholangiocarcinoma.

In a preferred embodiment, the prevention and/or treatment comprise(s) administrating to a patient a therapeutically effective amount of the antibody-drug conjugate of Formula (I) (preferably the antibody-drug conjugate of Formula (I-1)), the pharmaceutically acceptable salt, the stereoisomer or the metabolite thereof, or the solvate of the foregoing, preferably at a dose of 0.1-15 mg/kg body weight, preferably at a dose of 0.1-10 mg/kg, 0.2-8 mg/kg, and 0.3-6 mg/kg, and more preferably at a dose of 0.3 mg/kg, 1.2 mg/kg, 3.6 mg/kg, 4.8 mg/kg, 6 mg/kg, and 7.2 mg/kg.

In a preferred embodiment, a frequency of administration is once a day, twice a day, three times a day, once a week, once every two weeks, once every three weeks, once every four weeks or once a month, once every five weeks, or once every six weeks.

In a preferred embodiment, a route of administration may be oral administration, parenteral administration, or transdermal administration, and the parenteral administration includes, but is not limited to, intravenous injection, subcutaneous injection, and intramuscular injection.

In a preferred embodiment, the antibody-drug conjugate of Formula (I) (preferably the antibody-drug conjugate of Formula (I-1)), the pharmaceutically acceptable salt, the stereoisomer or the metabolite thereof, or the solvate of the foregoing is administered by injection, e.g., subcutaneous or intravenous injection. Before injection, the antibody-drug conjugate of Formula (I) (preferably the antibody-drug conjugate of Formula (I-1)), the pharmaceutically acceptable salt, the stereoisomer or the metabolite thereof, or the solvate of the foregoing needs to be formulated into an injectable form. A particularly preferred injectable form is an injection liquid or lyophilized powder for injection, comprising the antibody-drug conjugate of Formula (I) (preferably the antibody-drug conjugate of Formula (I-1)), the pharmaceutically acceptable salt, the stereoisomer or the metabolite thereof, or the solvate of the foregoing, as well as a buffering agent, a stabilizing agent, a pH regulating agent, and optionally a surfactant, wherein the buffering agent can be selected from one or more of acetates, citrates, succinates, and phosphates; the stabilizing agent can be selected from a group consisting of saccharides and amino acids, preferably disaccharides, such as sucrose, lactose, mycose, and maltose; the surfactant can be selected from a group consisting of polyoxyethylene hydrogenated castor oil, glycerin fatty acid ester, and polyoxyethylenesorbitan fatty acid ester, and preferably the polyoxyethylenesorbitan fatty acid ester is polysorbate 20, 40, 60, or 80, most preferably polysorbate 20; and the pH regulating agent can be selected from one or more of sodium hydroxide, lithium hydroxide, and potassium hydroxide.

In a preferred embodiment, the patient has also received a pretreatment step prior to the prevention and/or treatment. Preferably, the pretreatment is performed using an antipyretic analgesic or an antihistamine drug.

In a preferred embodiment, the antipyretic analgesic is selected from a group consisting of acetaminophen, ibuprofen, and the like.

In a preferred embodiment, the antihistamine drug is selected from a group consisting of diphenhydramine, promethazine, and the like.

In a preferred embodiment, the patient has also received other treatments which include, but are not limited to, a surgical therapy, a radiotherapy, and a drug therapy.

In a preferred embodiment, the drug therapy includes, but is not limited to, an anti-HER2 therapy, a hormone therapy, and a chemotherapy.

In a preferred embodiment, the anti-HER2 therapy includes, but is not limited to, an anti-HER2 antibody drug, such as a monoclonal antibody, an antibody-drug conjugate (ADC), and a bispecific antibody, or a chemical drug targeting HER2, such as lapatinib, neratinib, afatinib, or varlitinib. Preferably, the drug targeting HER2 includes trastuzumab or pertuzumab or a biosimilar thereof, such as ABP980, GB221, MYL-1401O, CT-P6, EG12014, HD201, ONS-1050, PF-05280014, Ontruzant, or HLX02, or includes an antibody-drug conjugate with trastuzumab or pertuzumab or a biosimilar thereof as a targeting component, such as an antibody-cytotoxic drug conjugate obtained by conjugation of the targeting component to DM1, DM4, MMAE, or MMAF, e.g., T-DM1.

In a preferred embodiment, the hormone includes, but is not limited to, estrogen receptor (ER) blockers such as tamoxifen, toremifene, fulvestrant, letrozole and anastrozole, ER modulators, or aromatase inhibitors, etc.

In a preferred embodiment, the chemotherapy drug includes, but is not limited to, paclitaxel, paclitaxel-albumin, docetaxel, gemcitabine, capecitabine, tegafur, carboplatin, vinorelbine, cyclophosphamide, and pharmorubicin, etc.

In a preferred embodiment, after the patient is treated with the antibody-drug conjugate of Formula (I), the pharmaceutically acceptable salt, the stereoisomer or the metabolite thereof, or the solvate of the foregoing, an incidence of thrombocytopenia in the patient is 10% or lower, preferably 8% or lower, and more preferably 5% or lower; and an incidence of thrombocytopenia of grade III and above is 5% or lower, preferably 3% or lower, more preferably 1% or lower, and most preferably 0;
and/or
after the patient is treated with the antibody-drug conjugate of Formula (I), the pharmaceutically acceptable salt, the stereoisomer or the metabolite thereof, or the solvate of the foregoing, an incidence of neutropenia in the patient is 20% or lower, preferably 10% or lower, more preferably 8% or lower, and further preferably 5% or lower; and an incidence of neutropenia of grade III and above is 5% or lower, preferably 3% or lower, more preferably 1% or lower, and most preferably 0;
and/or
after the patient is treated with the antibody-drug conjugate of Formula (I), the pharmaceutically acceptable salt, the stereoisomer or the metabolite thereof, or the solvate of the foregoing, an incidence of leukopenia in the patient is 10% or lower, preferably 8% or lower, more preferably 5% or lower, and further preferably 3% or lower; and an incidence of leukopenia of grade III and above is 5% or lower, preferably 3% or lower, more preferably 1% or lower, and most preferably 0;
and/or
after the patient is treated with the antibody-drug conjugate of Formula (I), the pharmaceutically acceptable salt, the stereoisomer or the metabolite thereof, or the solvate of the foregoing, an incidence of anemia in the patient is 30% or lower, preferably 25% or lower, and more preferably 20% or lower; and an incidence of anemia of grade III and above is 10% or lower, preferably 8% or lower, more preferably 5% or lower, and further preferably 3% or lower;
and/or
after the patient is treated with the antibody-drug conjugate of Formula (I), the pharmaceutically acceptable salt, the stereoisomer or the metabolite thereof, or the solvate of the foregoing, an incidence of nausea in the patient is 30% or lower, preferably 25% or lower, more preferably 20% or lower, and further preferably 15% or lower; and an incidence of nausea of grade III and above is 5% or lower, preferably 3% or lower, more preferably 1% or lower, and most preferably 0;
and/or
after the patient is treated with the antibody-drug conjugate of Formula (I), the pharmaceutically acceptable salt, the stereoisomer or the metabolite thereof, or the solvate of the foregoing, an incidence of vomiting in the patient is 30% or lower, preferably 20% or lower, more preferably 15% or lower, and further preferably 10% or lower; and an incidence of vomiting of grade III and above is 5% or lower, preferably 3% or lower, more preferably 1% or lower, and most preferably 0;
and/or
after the patient is treated with the antibody-drug conjugate of Formula (I), the pharmaceutically acceptable salt, the stereoisomer or the metabolite thereof, or the solvate of the foregoing, an incidence of diarrhea in the patient is 30% or lower, preferably 20% or lower, more preferably 15% or lower, further preferably 10% or lower, and still further preferably 8% or lower; and an incidence of diarrhea of grade III and above is 5% or lower, preferably 3% or lower, more preferably 1% or lower, and most preferably 0;
and/or
after the patient is treated with the antibody-drug conjugate of Formula (I), the pharmaceutically acceptable salt, the stereoisomer or the metabolite thereof, or the solvate of the foregoing, an incidence of pulmonary toxicity in the patient is 5% or lower, preferably 3% or lower, more preferably 1% or lower, and most preferably 0; and an incidence of pulmonary toxicity of grade III and above is 5% or lower, preferably 3% or lower, more preferably 1% or lower, and most preferably 0.

### DETAILED DESCRIPTION OF THE INVENTION

Unless otherwise defined, all the terms used herein have the same meaning as that commonly understood by one of ordinary skill in the art. Relevant definitions and terms can be found in e.g., Current Protocols in Molecular Biology (Ausubel).

All the documents mentioned in the description are incorporated herein by reference in their entirety.

The term “HER2” refers to human HER2 protein having a natural sequence (Genbank accession number X03363, see, e.g., Semba et al., 1985, PNAS, 82: 6497-6501; and Yamamoto et al., 1986, Nature, 319: 230-234), and functional derivatives thereof, e.g., amino acid sequence variants.

The natural sequence of HER2 as used herein can be isolated from nature, or can be produced by a recombinant DNA technology, a chemical synthesis, or a combination thereof.

The term “antibody” as used herein is interpreted in its broadest sense, including complete monoclonal antibodies, polyclonal antibodies, and multi-specific antibodies formed from at least two complete antibodies (e.g., bispecific antibodies), so long as they exhibit the desired biological activity.

The term “monoclonal antibody” as used herein refers to an antibody from a population of substantially homogeneous antibodies, i.e., various antibodies constituting the population are identical except for possible naturally occurring mutations that may be present in minor amounts. The monoclonal antibodies are highly specific to a single antigenic determinant (epitope), and in contrast, polyclonal antibodies comprise different antibodies directed against different determinants (epitopes). In addition to specificity, the monoclonal antibodies are advantageous in that they may not be contaminated by other antibodies during synthesis. The modifier “monoclonal” here indicates that the antibody is characterized by deriving from a substantially homogeneous population of antibodies, but should not be construed as being prepared by a particular method.

The monoclonal antibodies as used herein specifically comprise chimeric antibodies in which a portion of the heavy and/or light chain is identical or homologous to antibodies of a certain species, a certain class, or a certain subclass, while the remainders are identical or homologous to antibodies of another species, another class, or another subclass, so long as they exhibit the desired biological activity (see, e.g., U.S. Pat. No. 4,816,567; and Morrison et al., 1984, PNAS, 81: 6851-6855).

Chimeric antibodies that can be used in the present invention include primatized antibodies comprising variable region antigen-binding sequences from a non-human primate (e.g., old world monkey, orangutan, etc.) and human constant region sequences.

The term “antibody fragment” refers to a portion of an antibody, preferably an antigen-binding region or a variable region. Examples of antibody fragments include: Fab, Fab′, F(ab′)2, and Fv fragments; diabodies; linear antibodies; and single-chain antibody molecules.

The term “bispecific antibody”, also known as “bifunctional antibody conjugate”, refers to a conjugate formed by a first antibody (fragment) and a second antibody (fragment) through a linker, and the conjugate retains the activity of the respective antibodies and therefore has dual functionality and dual specificity.

The term “multi-specific antibody” includes, for example, tri- and tetra-specific antibodies, and the former is an antibody having three different types of antigen-binding specificity, while the latter is an antibody having four different types of antigen-binding specificity.

The term “complete antibody” refers to an antibody comprising an antigen-binding variable region, as well as a light chain constant region (CL) and heavy chain constant regions (CH1, CH2 and CH3). The constant regions may be natural sequences (e.g., human natural constant region sequences) or amino acid sequence variants thereof. The complete antibody is preferably a complete antibody having one or more effector functions.

“Humanized” forms of non-human (e.g., mouse) antibodies refer to chimeric antibodies that contain a minimal amount of non-human immunoglobulin sequences. In most of the humanized antibodies, the hypervariable region residues of the immunoglobulins of human recipients are replaced with non-human (e.g., mouse, rat, rabbit, or non-human primate) hypervariable region residues (donor antibodies) with all specificity, affinity and functions. In some embodiments, the framework region (FR) residues of the human immunoglobulin are also replaced with non-human residues. Furthermore, humanized antibodies may also comprise residues that are not found in a recipient antibody or in a donor antibody. These modifications are made to further optimize the performance of the antibody. A humanized antibody generally comprises at least one, typically two variable regions, in which all or almost all of the hypervariable loops correspond to those of the non-human immunoglobulin, and all or almost all of the FRs are human immunoglobulin sequences. A humanized antibody can also comprise at least a portion of an immunoglobulin constant region (Fc, typically Fc of a human immunoglobulin). For details, see, e.g., Jones et al., 1986, Nature, 321: 522-525; Riechmann et al., 1988, Nature, 332: 323-329; and Presta, 1992, Curr. Op. Struct. Bwl 2: 593-596.

Depending on the amino acid sequence of the heavy chain constant region, the complete antibodies can be assigned to different “classes”. The five major classes are IgA, IgD, IgE, IgG, and IgM, and several of them may be further divided into “subclasses” (isotypes), e.g., IgG1, IgG2, IgG3, IgG4, IgA1, and IgA2. Different classes of the heavy chain constant regions of antibodies are known as α, δ, ε, γ, and μ, respectively. Different classes of the subunit structures and the three-dimensional configurations of immunoglobulins are structures and configurations well known in the art.

Herein, the amino acid substitutions in antibodies are substitutions with L-amino acids in most cases, but are not limited thereto. In some embodiments, the peptide chains of an antibody may comprise one or more D-amino acids. Peptides containing D-amino acids are thought to be more stable and less prone to degradation in oral cavity, gut or plasma than peptides containing L-amino acids alone.

The monoclonal antibodies used in the present invention can be produced by various methods. For example, the monoclonal antibodies used in the present invention can be obtained by a hybridoma method using various species (including cells of mice, hamsters, rats and human) (see, e.g., Kohler et al., 1975, Nature, 256: 495), or by a recombinant DNA technology (see, e.g., U.S. Pat. No. 4,816,567), or by isolation from a phage antibody library (see, e.g., Clackson et al., 1991, Nature, 352: 624-628; and Marks et al., 1991, Journal of Molecular Biology, 222: 581-597).

The anti-HER2 antibody in the present invention is preferably an anti-human HER2 antibody. Preferably, the CDR1, CDR2 and/or CDR3 in the heavy and light chains of the anti-human HER2 antibody are the CDR1, CDR2 and/or CDR3 in the heavy and light chains of trastuzumab, respectively. The anti-human HER2 antibody can be a humanized antibody or a fully human antibody.

More preferably, the anti-HER2 antibody used in the present invention is a murine anti-human HER2 antibody 4D5 shown in FIG. 1 of U.S. Pat. No. 5,821,337.

Particularly preferably, the anti-HER2 antibody used in the present invention is trastuzumab, the sequence of which has been disclosed in, e.g., CN 103319599A. The Lys at the end of the heavy chain of trastuzumab is prone to deletion, but such a deletion does not affect biological activity (see Dick, L. W. et al., Biotechnol. Bioeng., 100: 1132-1143). The above trastuzumab and a sequence thereof in which the Lys at the end of the heavy chain is deleted or a fragment thereof are included within the scope of the trastuzumab of the present invention.

The complementarity determining region (CDR) sequences of trastuzumab used in the resent invention are as follows:

| | CDR1 | CDR2 | CDR3 |
|---|---|---|---|
| Heavy Chain | DTYIH | RIYPTNGYTRYADSVKG | WGGDGFYAMDY |
| | (SEQ ID NO. 1) | (SEQ ID NO. 2) | (SEQ ID NO. 3) |
| Light Chain | RASQDVNTAVA | SASFLYS | QQHYTTPPT |
| | (SEQ ID NO. 4) | (SEQ ID NO. 5) | (SEQ ID NO. 6) |

The heavy-chain sequences of trastuzumab (SEQ ID NO. 7) used in the present invention are as follows:

| | |
|---|---|
| EVQLVESGGG LVQPGGSLRL SCAASGFNIK DTYIHWVRQA PGKGLEWVAR | 50 |
| IYPTNGYTRY ADSVKGRFTI SADTSKNTAY LQMNSLRAED TAVYYCSRWG | 100 |
| GDGFYAMDYW GQGTLVTVSS ASTKGPSVFP LAPSSKSTSG GTAALGCLVK | 150 |
| DYFPEPVTVS WNSGALTSGV HTFPAVLQSS GLYSLSSVVT VPSSSLGTQT | 200 |
| YICNVNHKPS NTKVDKKVEP KSCDKTHTCP PCPAPELLGG PSVFLFPPKP | 250 |
| KDTLMISRTP EVTCVVVDVS HEDPEVKFNW YVDGVEVHNA KTKPREEQYN | 300 |
| STYRVVSVLT VLHQDWLNGK EYKCKVSNKA LPAPIEKTIS KAKGQPREPQ | 350 |
| VYTLPPSREE MTKNQVSLTC LVKGFYPSDI AVEWESNGQP ENNYKTTPPV | 400 |
| LDSDGSFFLY SKLTVDKSRW QQGNVFSCSV MHEALHNHYT QKSLSLSPGK. | 450 |

The light-chain sequences of trastuzumab (SEQ ID NO. 8) used in the present invention are as follows:

| | |
|---|---|
| DIQMTQSPSS LSASVGDRVT ITCRASQDVN TAVAWYQQKP GKAPKLLIYS | 50 |
| ASFLYSGVPS RFSGSRSGTD FTLTISSLQP EDFATYYCQQ HYTTPPTFGQ | 100 |
| GTKVEIKRTV AAPSVFIFPP SDEQLKSGTA SVVCLLNNFY PREAKVQWKV | 150 |
| DNALQSGNSQ ESVTEQDSKD STYSLSSTLT LSKADYEKHK VYACEVTHQG | 200 |
| LSSPVTKSFN RGEC. | 214 |

The term “cytotoxic drug” as used herein refers to a substance that inhibits or prevents the function of cells and/or causes destruction of cells.

The antibody-drug conjugate of the present invention can be in the form of a pharmaceutically acceptable salt, or a stereoisomer, or a metabolite, or a solvate, and the salt, the stereoisomer, or the metabolite can also be in the form of a solvate.

The term “pharmaceutically acceptable salts” refers to salts that retain the bioavailability and nature of a compound, and are biologically or otherwise desirable for use as drugs. In many cases, the antibody-drug conjugate of the present invention can form an acid addition salt and/or base addition salt via an amino group and/or a carboxyl group or other similar groups therein.

Pharmaceutically acceptable acid addition salts can be those formed with inorganic or organic acids. The inorganic acids include, e.g., hydrochloric acid, hydrobromic acid, sulfuric acid, nitric acid, phosphorus acid, etc. The organic acids include, e.g., acetic acid, propionic acid, hydroxyacetic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, etc.

Pharmaceutically acceptable base addition salts can be those formed with inorganic or organic bases. The salts formed with inorganic bases include, e.g., sodium salts, potassium salts, lithium salts, ammonium salts, calcium salts, magnesium salts, iron salts, zinc salts, copper salts, manganese salts, aluminum salts, etc., wherein the ammonium salts, potassium salts, sodium salts, calcium salts, and magnesium salts are particularly preferred. The organic bases include, e.g., primary amines, secondary amines, tertiary amines, substituted amines (including naturally occurring substituted amines), cyclic amines, basic ion exchange resins, etc. Specific examples of the organic bases are isopropylamine, trimethylamine, diethylamine, N-ethylethanamine, tripropylamine, and ethanolamine.

The term “stereoisomer” refers to an isomer formed due to the existence of at least one asymmetric center. In a compound with one or more asymmetric centers, a racemate, a racemic mixture, a single enantiomer, a diastereomeric mixture, and a single diastereomer can exist. Specific individual molecules may be present as geometric (cis-/trans-) isomers. Unless otherwise specified, when a disclosed compound exhibits no clear stereochemistry in its name or structure and has one or more asymmetric centers, it should be understood that all the possible stereoisomers of the compounds are contemplated.

The term “solvate” refers to a solvate formed by association of one or more solvent molecules and any of the antibody-drug conjugate of Formula (I) or a pharmaceutically acceptable salt or isomer thereof. The term “solvate” includes a hydrate (e.g., a hemihydrate, a monohydrate, a dihydrate, a trihydrate, a tetrahydrate, and similar hydrates).

The term “metabolite” refers to a substance generated via oxidation, reduction, hydrolysis, amidation, deamidation, esterification and/or enzymolysis in vivo upon administration.

The term “treatment” as used herein refers to a method implemented to obtain beneficial or desired clinical results. For the purposes of the present invention, the beneficial or desired clinical results include, but are not limited to, alleviating symptoms, narrowing down the range of a disease, stabilizing (i.e., stopping worsening) the state of a disease, delaying or slowing down the development of a disease, improving or alleviating the state of a disease, and relieving symptoms (no matter whether in part or in whole), no matter whether detectable or undetectable. Furthermore, the term “treatment” may also refer to prolonging the survival time compared with the expected survival time (if the treatment is not received).

As used herein, the methods for determining the HER2 expression, amplification levels and mutations in a patient and relevant technologies thereof are known in the art. For example, a detection for HER2 includes, but is not limited to, a detection for HER2 protein by IHC, a detection for HER2 gene amplification by FISH technology, and a detection for HER2 gene mutation by NGS technology.

The term “HER2 low-expression” as used herein generally refers to an HER2 expression level of IHC 1+ or IHC 2+/FISH negative (i.e., IHC 2+ while FISH test negative) in a clinical test. The terms “HER2 over-expression”, “HER2 overexpression” and “HER2 positive” are used interchangeably, and generally refer to an HER2 expression level of IHC 2+/FISH positive (i.e., IHC 2+ while FISH test positive) or IHC 3+ in a clinical test.

In some tumors, IHC 2+(FISH negative or positive) cancers may also be considered as HER2 positive cancers, e.g., urothelial cancer. Herein, FISH negative means that a FISH test result shows no amplification of HER2 genes, and FISH positive means that a FISH test result shows amplification of HER2 genes.

The term “HER2 mutation” as used herein generally refers to an HER2 gene mutation detected by NGS.

The term “progression of disease” or “disease progression” as used herein means that by taking the minimum value of the sum of the diameters of all the target lesions in an entire study (if the sum of baselines is the minimum value in the study, the sum of the baselines is included) as reference, the sum of the diameters of the target lesions increases by at least 20%, and the sum of the diameters has an absolute increase of at least 5 mm; or it refers to the appearance of one or more new lesions.

The term “therapeutically effective amount” or “effective amount” as used herein refers to an amount of anti-HER2-antibody-drug conjugates (anti-HER2-ADCs), when administered alone or in combination to cells, tissues or a subject, to effectively prevent or retard a disease or a condition to be treated. The term “therapeutically effective amount” further refers to an amount of an antibody-drug conjugate (ADC) and/or an antibody or a fragment thereof that is sufficient to cause easing of a symptom, and the easing of the symptom is, e.g., treating, curing, or easing a related medical state, or increasing the treatment rate, cure rate, or easing rate of the symptom. An effective amount for a specific subject may vary depending on various factors, e.g., the disease to be treated, the general health condition of the patient, the method, route and dosage of administration, and the severity of an adverse effect. The effective amount may be a maximum dose or a dosing regimen that can avoid a significant adverse effect or a toxic effect. The therapeutically effective amount will alleviate a symptom typically by at least 10%, typically by at least 20%, by at least about 30%, by at least 40%, or by at least 50%.

The term “individual”, “subject” or “patient” as used herein refers to an animal serving as an object of treatment, observation, or experiment. Just by way of example, a patient may be (but not limited to) a mammal, including (but not limited to) human.

As used herein, the term “complete response (CR)”, “partial response (PR)”, “stable disease (SD)”, and “progressive disease (PD)” are defined by the following methods:

When the disease is a solid tumor, the efficacy for the solid tumor is evaluated according to the following criteria (see New response evaluation criteria in solid tumors: Revised RECIST guideline (version 1.1), E. A. Eisenhauer et al., EUROPEAN JOURNAL OF CANCER, 45 (2009), pp. 228-247) to be “complete response (CR)”, “partial response (PR)”, “stable disease (SD)”, and “progressive disease (PD)”. The target lesions are specifically evaluated as follows:

Complete response (CR): All the target lesions disappear, and every pathological lymph node (including target and non-target nodes) must have a reduction in short diameter to <10 mm. There are no new lesions.

Partial response (PR): The sum of the diameters of target lesions (the short diameter of a lymph node is taken into account) decreases by at least 30% from a baseline level. Non-target lesions have no significant progression, and there are no new lesions.

Stable disease (SD): The reduction of target lesions does not reach the PR level, and the increase of target lesions does not reach the PD level, that is, between the PR and PD levels. Across the study, the minimum value of the sum of the diameters can be used as reference.

Progress of disease (PD): By taking the minimal value of the sum of the diameters of the measured target lesions across the test study as reference, the sum of the diameters relatively increases by at least 20% (if a baseline measurement value is minimum, the baseline value is taken as reference). In addition, the at least 5 mm increase in the absolute value of the sum of the diameters of the measured target lesions must be met (the appearance of one or more new lesions is also considered as PD).

An adverse event (AE) of a drug is evaluated with reference to NCI CTCAE (version 4.03/version 5.0). General grades of each AE of the drug are as follows:

### Version 4.03:

Grade 1: Mild; asymptomatic or mild symptoms; clinical or diagnostic observations only; intervention not needed.
Grade 2: Moderate; minimal, local or noninvasive intervention needed; limiting age-appropriate instrumental activities of daily living*.
Grade 3: Severe or medically significant but not immediately life-threatening; resulting in hospitalization or prolongation of hospitalization; disabling; limiting personal activities of daily living **.
Grade 4: Life-threatening consequences; urgent intervention needed.
Grade 5: Death.
*Instrumental activities of daily living refer to preparing meals, shopping for groceries or clothes, using the telephone, managing money, etc.
**Personal activities of daily living refer to bathing, dressing and undressing, feeding self, using the toilet, taking medications, and not bedridden.

### Version 5.0:

Grade 1: Mild; asymptomatic or mild symptoms; clinical or diagnostic observations only; intervention not needed.
Grade 2: Moderate; minimal, local or noninvasive intervention needed; limiting age-appropriate instrumental activities of daily living*.
Grade 3: Severe or medically significant but not immediately life-threatening; hospitalization or prolongation of hospitalization needed; disabling; limiting self-care activities of daily living **.
Grade 4: Life-threatening consequences; urgent intervention needed.
Grade 5: Death related to AE.
*Instrumental activities of daily living refer to preparing meals, shopping for groceries or clothes, using the telephone, managing money, etc.
**Self-care activities of daily living refers to bathing, dressing and undressing, feeding self, using the toilet, taking medications, and not bedridden.

The present invention will be described in the following examples, and the examples are merely intended to illustrate rather than limit the present invention.

### EXAMPLES

The meanings of abbreviations used hereinafter are as shown in the following table:

| | |
|---|---|
| DMF | dimethylformamide |
| DIC | diisopropylcarbodiimide |
| HOAt | 1-hydroxy-7-azabenzotriazole |
| EtOAc | ethyl acetate |
| DIEA | diisopropylethylamine |
| HATU | 2-(1H-7-azabenzotriazol-1-yl)-1,1,3,3-tetramethyluronium |
| | hexafluorophosphate |
| PyBOP | (1H-benzotriazol-1-yloxy)tripyrrolidinophosphonium |
| | hexafluorophosphate |
| HOBT | 1-hydroxybenzotriazole |
| LiOH | lithium hydroxide |
| DCM | dichloromethane |
| EDCI | 1-ethyl-3-(3-dimethylaminopropyl)carbodiimide |
| NHS | N-hydroxysuccinimide |
| DMA | N,N-dimethylacetamide |
| HIC | hydrophobic interaction chromatography |
| HPLC | high performance liquid chromatography |
| UPLC | ultra performance liquid chromatography |
| THF | tetrahydrofuran |
| EtOAc | ethyl acetate |

### Preparation Example 1. Preparation of Antibody-Drug Conjugate I-1

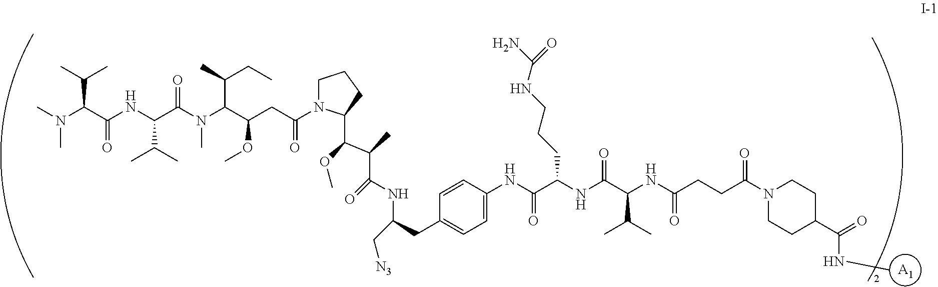

### Step a. Preparation of Intermediate D-1

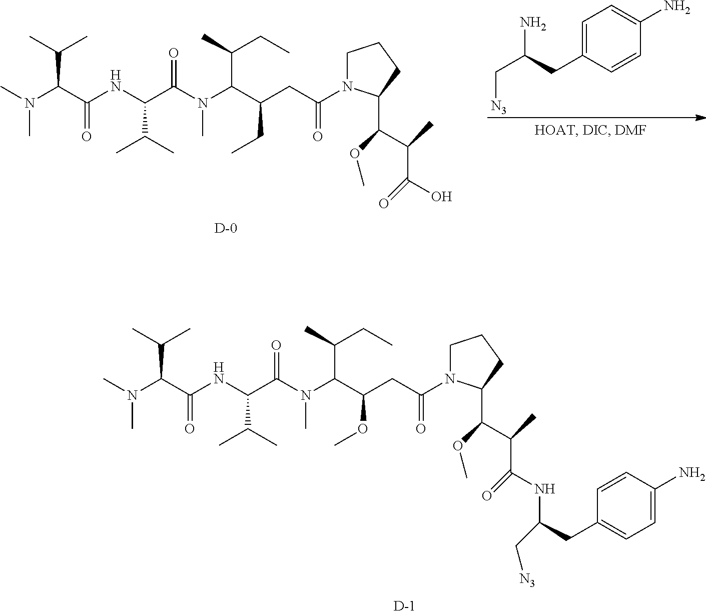

At room temperature, compound D-0 (1 mmol, 1 equivalent) was dissolved in DMF (50 mL), and DIC (1.1 equivalents), HOAt (1.1 equivalents) and 4-(3-azido-2-aminopropyl)aniline (1.5 equivalents) were added successively. The reaction mixture was stirred at room temperature for 8 hours, and then water (600 mL) and EtOAc (200 mL*3) were added. After extraction, the organic phase was collected, concentrated, and subjected to HPLC for purification to obtain intermediate D-1.

MS m/z (ESI): 773 [M+H]⁺.

### Step b. Preparation of Intermediate I-A-1

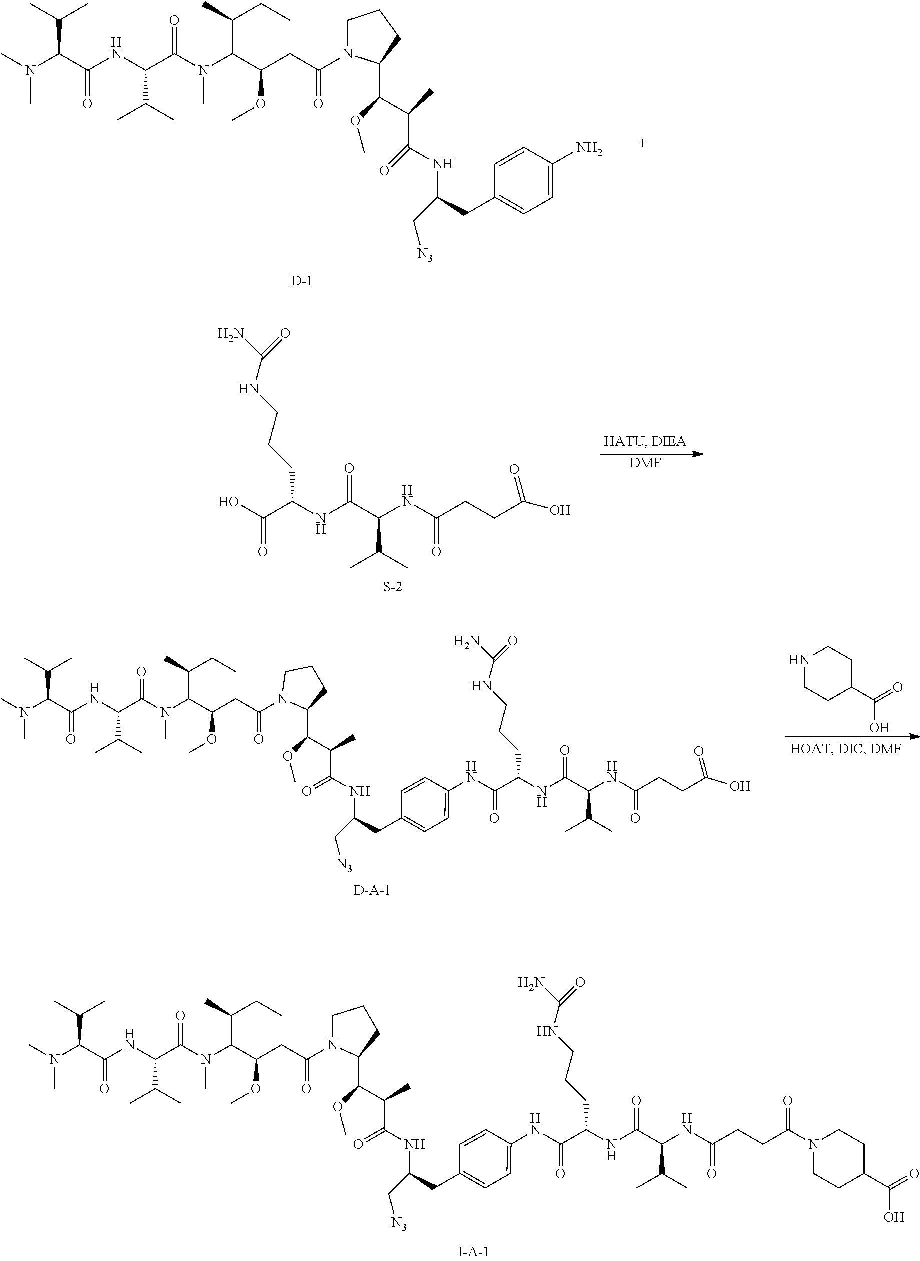

At room temperature, compound S-2 (0.1 mmol, 1 equivalent) was dissolved in DMF (50 mL), and DIEA (2 equivalents), HATU (1.05 equivalents) and the compound D-1 (2.0 equivalents) were added successively. After reaction at room temperature for 12 hours, water (300 mL) and EtOAc (100 mL*3) were added. After extraction, the organic phase was collected, concentrated, and subjected to HPLC for purification to obtain intermediate D-A-1.

At room temperature, the compound D-A-1 (0.1 mmol, 1 equivalent) was dissolved in DMF (5 mL), and DIC (1.1 equivalents), HOAt (1.1 equivalents) and piperidine-4-carboxylic acid (1.2 equivalents) were added successively. The reaction mixture was stirred at room temperature for 6 hours, and then water (60 mL) and EtOAc (20 mL*3) were added. After extraction, the organic phase was collected, concentrated, and subjected to HPLC for purification to obtain intermediate I-A-1.

MS m/z (ESI): 1240 [M+H]⁺.

Alternatively,

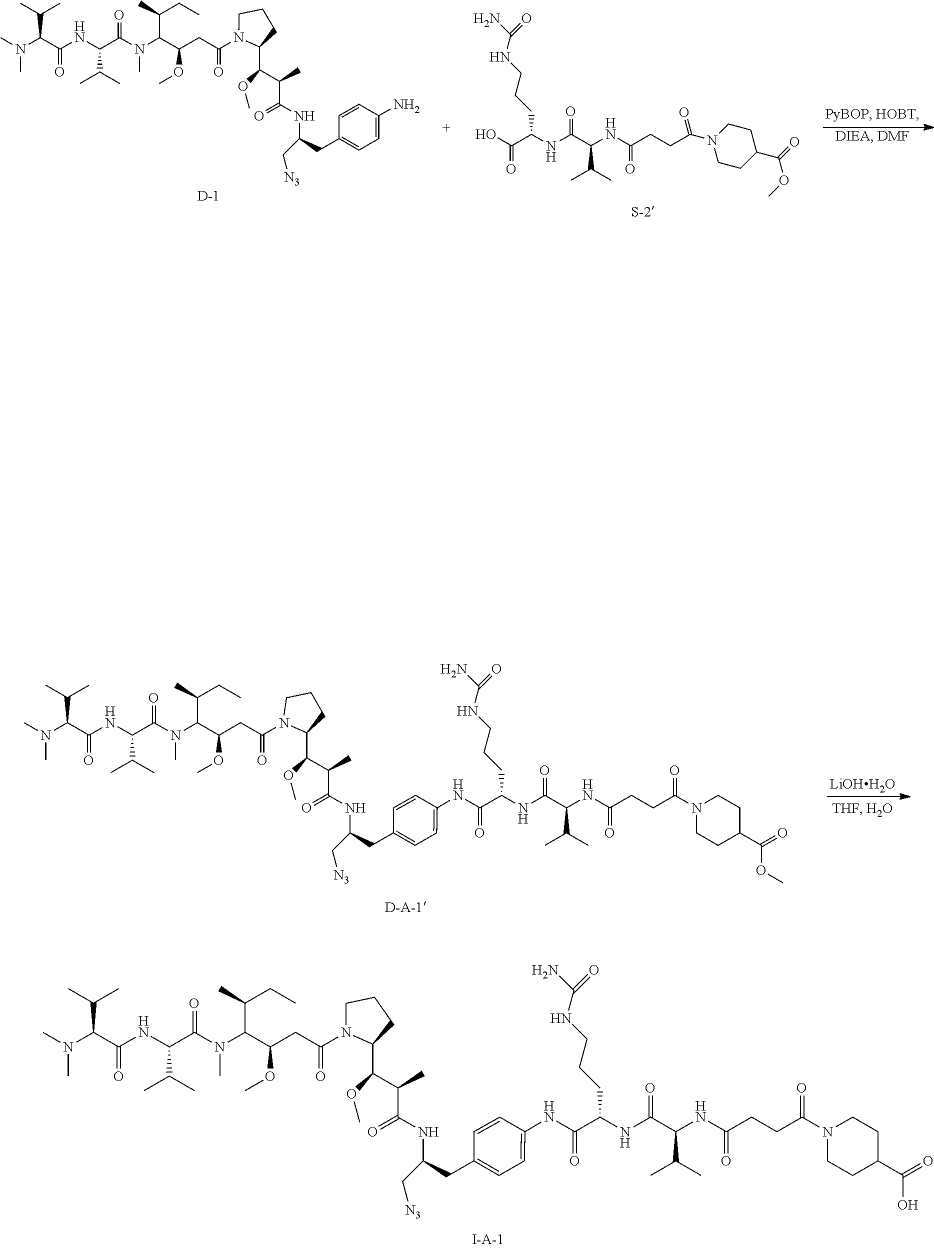

In an ice-water bath, Compound S-2′ (0.1 mmol, 1.0 equivalent) was dissolved in DMF (10 mL), and DIEA (2.0 equivalents), PyBOP (1.0 equivalent), HOBT (1.0 equivalent) and the compound D-1 (0.2 mmol, 2.0 equivalents) were added successively. After reaction at room temperature for 12 hours, water (30 mL) and EtOAc (10 mL*3) were added. After extraction, the organic phase was collected, concentrated, and subjected to HPLC for purification to obtain intermediate D-A-1′.

At room temperature, the compound D-A-1′ (0.05 mmol, 1.0 equivalent) was dissolved in THF/H₂O (6 mL, v:v=5:1), and LiOH monohydrate (3.0 equivalents) was added. The reaction mixture was stirred at room temperature for 16 hours, and then subjected to separation and purification to obtain intermediate I-A-1.

MS m/z (ESI): 1240 [M+H]⁺.

### Step c. Preparation of Intermediate I-B-1

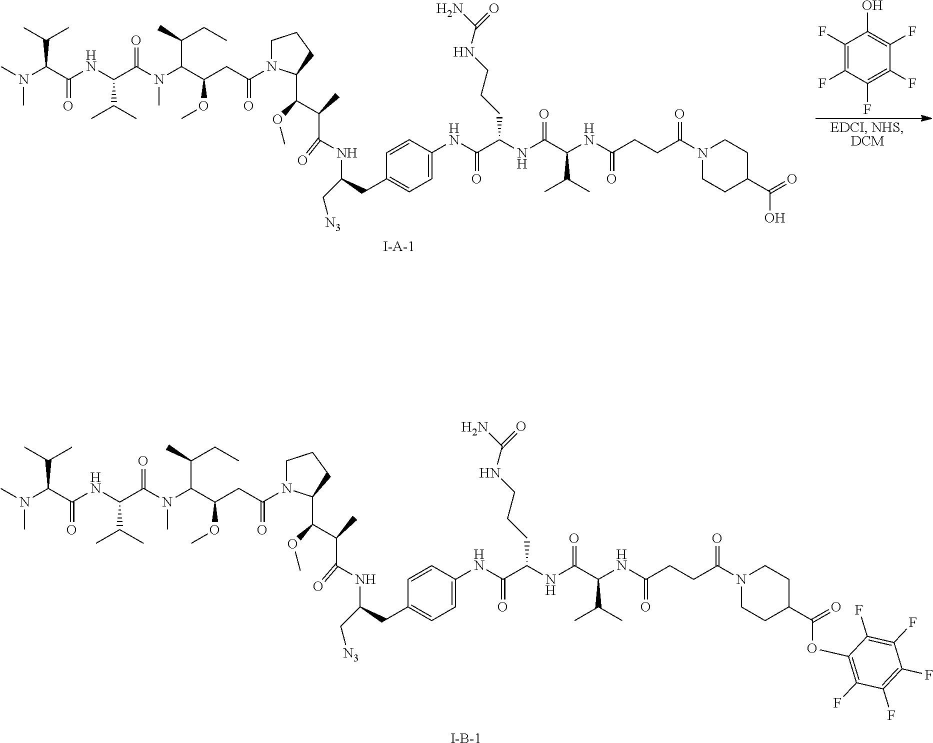

At room temperature, the compound I-A-1 (0.1 mmol, 1 equivalent) was dissolved in DCM (50 mL), and EDCI (1.5 equivalents), NHS (1.5 equivalents) and pentafluorophenol (2.0 equivalents) were added successively. The reaction proceeded for 18 hours at room temperature. The reaction mixture was washed successively with water (30 mL), a 10% (w/v) aqueous citric acid solution (20 mL) and a saturated aqueous sodium chloride solution (20 mL), and the organic phase was collected, concentrated and subjected to HPLC for purification to obtain intermediate I-B-1.

MS m/z (ESI): 1406 [M+H]⁺.

### Step d. Synthesis of Crude Antibody-Drug Conjugate I-1

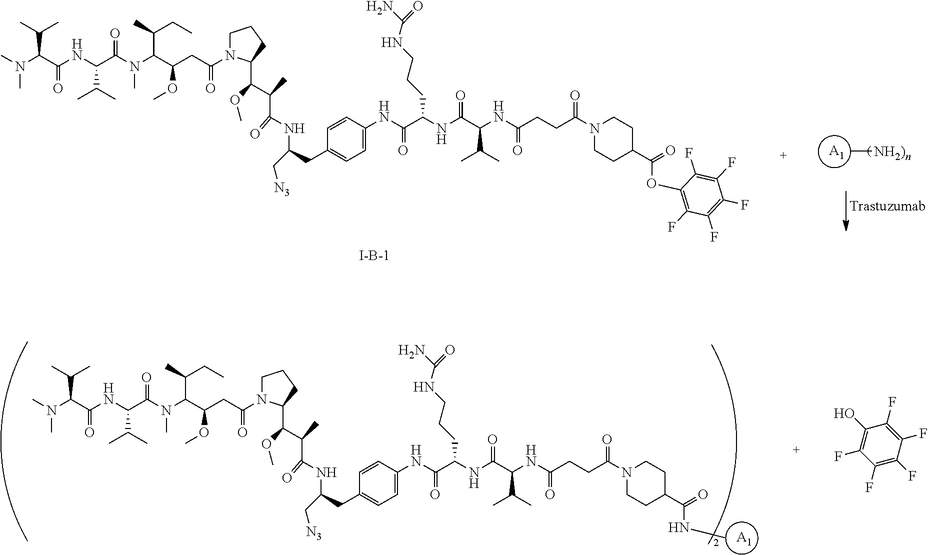

Crude I-1 (wherein n=1, 2, 3, 4)

To 1 ml solution of 10-20 mg/ml of Trastuzumab prepared in PBS buffer (pH=7.4), 4-6 folds molar amount of compound I-B-1 dissolved in DMA was added. The reaction proceeded under gentle stirring for 2-6 hours at a temperature in the range of 2-40° C., and was monitored by HIC-HPLC, to obtain crude Antibody-Drug Conjugate I-1.

### Hic-HPLC Conditions:

Column: Tosoh TSKgel Butyl-NPR, 4.6×100 mm
Mobile phase A: 1.5 M aqueous ammonium sulfate solution
Mobile phase B: 25 mM aqueous sodium phosphate solution, pH=7.0, and 25% (v/v) aqueous isopropanol solution
Flow rate: 0.5 ml/min
Gradients: 0-2 min: 17% mobile phase B+83% mobile phase A
   2-15 min: 17%-40% mobile phase B+83%-60% mobile phase A
   15-15.1 min: 40%-70% mobile phase B+60%-30% mobile phase A
   15.1-17 min: 70% mobile phase B+30% mobile phase A.

### Step e. Purification of Crude Antibody-Drug Conjugate I-1

The crude Antibody-Drug Conjugate I-1 obtained in step d was purified by HIC, then desalted by buffer change, and concentrated by ultrafiltration to obtain Antibody-Drug Conjugate I-1, which was an antibody-drug conjugate obtained by conjugation of one molecule of antibody to two molecules of drugs, and the product was pure.

### Hic Conditions:

Packing material: GE packing material (Pheynl HP)
Mobile phase A: 1.5 M aqueous ammonium sulfate solution, and 25 mM aqueous disodium hydrogen phosphate solution, pH 7.0
Mobile phase B: 25 mM aqueous disodium hydrogen phosphate solution, pH=7.0, and 10% aqueous isopropanol solution
Flow rate: 1.0 ml/min
Elution conditions: elution in 20 CV with 0%-40% mobile phase B; elution in 30 CV with 40%-100% mobile phase B, and collection in different tubes.

A clinical trial study on the compound I-1 included patients with unresectable locally advanced or metastatic solid tumors with HER2 expression (defined as IHC 1+) or HER2 amplification or mutations.

### Example 1: Efficacy of the Antibody-Drug Conjugate on the Treatment of Gallbladder Cancer

One patient suffering from gallbladder cancer with an HER2 expression level of IHC 3+ received a treatment with the compound I-1 alone at a dose of 3.6 mg/kg by intravenous injection, once every three weeks, and efficacy evaluation was performed after 9 weeks. After the treatment with the compound I-1, the efficacy was evaluated to be PR (partial response), demonstrating the efficacy of the compound I-1 in the treatment of gallbladder cancer.

### Example 2: Efficacy of the Antibody-Drug Conjugate on the Treatment of Parotid Gland Carcinoma

Two patients suffering from parotid gland carcinoma with respective HER2 expression levels of IHC 2+ and IHC 3+ received a treatment with the compound I-1 alone at a dose of 3.6 mg/kg by intravenous injection, once every three weeks, and efficacy evaluation was performed after 9 weeks. After the treatment with the compound I-1, the efficacy was evaluated to be PR (partial response) for both, demonstrating the efficacy of the compound I-1 in the treatment of parotid gland carcinoma.

Another patient suffering from salivary duct carcinoma with an HER2 expression level of IHC 1+ received a treatment with the compound I-1 alone at a dose of 4.8 mg/kg by intravenous injection, once every three weeks, and efficacy evaluation was performed after 9 weeks. After the treatment with the compound I-1, the efficacy was evaluated to be PR (partial response), demonstrating the efficacy of the compound I-1 in the treatment of salivary duct carcinoma.

### Example 3: Efficacy of the Antibody-Drug Conjugate on the Treatment of Upper Gastrointestinal Cancer

One patient suffering from esophageal adenocarcinoma with HER2 amplification received a treatment with the compound I-1 alone at a dose of 3.6 mg/kg by intravenous injection, once every three weeks, and efficacy evaluation was performed after 9 weeks. After the treatment with the compound I-1, the efficacy was evaluated to be PR (partial response), demonstrating the efficacy of the compound I-1 in the treatment of esophageal adenocarcinoma.

One patient suffering from bile duct carcinoma in upper gastrointestinal tract with HER2 mutation received a treatment with the compound I-1 alone at a dose of 3.6 mg/kg by intravenous injection, once every three weeks, and efficacy evaluation was performed after 9 weeks. After the treatment with the compound I-1, the efficacy was evaluated to be SD (stable disease), demonstrating the efficacy of the compound I-1 in the treatment of bile duct carcinoma in upper gastrointestinal tract.

### Example 4: Efficacy of the Antibody-Drug Conjugate on the Treatment of Intrahepatic Cholangiocarcinoma

One patient suffering from intrahepatic cholangiocarcinoma with HER2 expression received a treatment with the compound I-1 alone at a dose of 4.8 mg/kg by intravenous injection, once every three weeks, and efficacy evaluation was performed after 9 weeks. After the treatment with the compound I-1, the efficacy was evaluated to be SD (stable disease), demonstrating the efficacy of the compound I-1 in the treatment of intrahepatic cholangiocarcinoma.

### Example 5: Efficacy of the Antibody-Drug Conjugate on the Treatment of Head and Neck Caner

Two patients suffering from head and neck squamous cell carcinoma with respective HER2 expression levels of IHC 2+ and IHC 3+ received a treatment with the compound I-1 alone at a dose of 4.8 mg/kg by intravenous injection, once every three weeks, and efficacy evaluation was performed after 9 weeks. After the treatment with the compound I-1, the efficacy was evaluated to be SD (stable disease) for both, demonstrating the efficacy of the compound I-1 in the treatment of head and neck cancer.

### Example 6: Efficacy of the Antibody-Drug Conjugate on the Treatment of Breast Caner

Four patients suffering from breast cancer with HER2 amplification received a treatment with the compound I-1 alone at a dose of 1.2 mg/kg by intravenous injection, once every three weeks, and efficacy evaluation was performed after 9 weeks. After the treatment with the compound I-1, the efficacy was evaluated to be SD (stable disease) for two patients.

Four patients suffering from breast cancer with an HER2 expression level of IHC 3+ received a treatment with the compound I-1 alone at a dose of 3.6 mg/kg by intravenous injection, once every three weeks, and efficacy evaluation was performed after 9 weeks. After the treatment with the compound I-1, the efficacy was evaluated to be PR (partial response) for 2 patients and SD (stable disease) for 2 patients.

25 Patients suffering from breast cancer (including 20 patients suffering from breast cancer with an HER2 expression level of IHC 3+, and 5 patients suffering from breast cancer with an HER2 expression level of IHC 2+/FISH positive) received a treatment with the compound I-1 alone at a dose of 4.8 mg/kg by intravenous injection, once every three weeks, and efficacy evaluation was performed after 2-24 weeks. After the treatment with the compound I-1, among 24 patients for whom the efficacy was evaluable, the efficacy was evaluated to be CR (complete response) for 2 patients, PR (partial response) for 16 patients, and SD (stable disease) for 3 patients.

25 Patients suffering from breast cancer with an HER2 expression level of IHC 3+, 10 patients suffering from breast cancer with an HER2 expression level of IHC 2+/FISH positive, and 13 patients suffering from breast cancer with an HER2 expression level of IHC 2+ and FISH negative or IHC 1+) each received a treatment with the compound I-1 alone at a dose of 6.0 mg/kg by intravenous injection, once every three weeks, and efficacy evaluation was performed after 4-12 weeks. After the treatment with the compound I-1, among 48 patients for whom the efficacy was evaluable, the efficacy was evaluated to be PR (partial response) for 26 patients and SD (stable disease) for 9 patients.

In conclusion, the compound I-1 exhibited good efficacy in treatment of patients suffering from breast cancer with HER2 low-expression, HER2 over-expression, or HER2 amplification.

### Example 7: Efficacy of the Antibody-Drug Conjugate on the Treatment of Gastroesophageal Junction Adenocarcinoma

One patient suffering from gastroesophageal junction adenocarcinoma with HER2 amplification received a treatment with the compound I-1 alone at a dose of 3.6 mg/kg by intravenous injection, once every three weeks, and efficacy evaluation was performed after 9 weeks. After the treatment with the compound I-1, the efficacy was evaluated to be SD (stable disease), demonstrating the efficacy of the compound I-1 in the treatment of gastroesophageal junction adenocarcinoma.

### Example 8: Efficacy of the Antibody-Drug Conjugate on the Treatment of Ovarian Cancer

One patient suffering from ovarian cancer with HER2 amplification/mutation received a treatment with the compound I-1 alone at a dose of 4.8 mg/kg by intravenous injection, once every three weeks, and efficacy evaluation was performed after 9 weeks. After the treatment with the compound I-1, the efficacy was evaluated to be SD (stable disease), demonstrating the efficacy of the compound I-1 in the treatment of ovarian cancer.

### Example 9: Efficacy of the Antibody-Drug Conjugate on the Treatment of Bladder Cancer

One patient suffering from bladder cancer with an HER2 expression level of IHC 3+ received a treatment with the compound I-1 alone at a dose of 4.8 mg/kg by intravenous injection, once every three weeks, and efficacy evaluation was performed after 9 weeks. After the treatment with the compound I-1, the efficacy was evaluated to be SD (stable disease), demonstrating the efficacy of the compound I-1 in the treatment of bladder cancer.

### Example 10: Efficacy of the Antibody-Drug Conjugate on the Treatment of Colorectal Cancer

Two patients suffering from colorectal cancer with an HER2 expression level of IHC 3+ received a treatment with the compound I-1 alone at a dose of 4.8 mg/kg by intravenous injection, once every three weeks, and efficacy evaluation was performed after 9 weeks. After the treatment with the compound I-1, the efficacy was evaluated to be PR (partial response), demonstrating the efficacy of the compound I-1 in the treatment of colorectal cancer.

### Example 11: Efficacy of the Antibody-Drug Conjugate on the Treatment of Lung Cancer

One patient suffering from lung cancer with HER2 amplification received a treatment with the compound I-1 alone at a dose of 3.6 mg/kg by intravenous injection, once every three weeks, and efficacy evaluation was performed after 9 weeks. After the treatment with the compound I-1, the efficacy was evaluated to be SD (stable disease).

One patient suffering from lung cancer with HER2 mutation received a treatment with the compound I-1 alone at a dose of 4.8 mg/kg by intravenous injection, once every three weeks, and efficacy evaluation was performed after 9 weeks. After the treatment with the compound I-1, the efficacy was evaluated to be PR (partial response).

In conclusion, the compound I-1 exhibited good efficacy in the treatment of lung cancer with HER2 amplification or mutation.

### Example 12: Pharmacokinetic Study

The compound I-1 was administered at doses of 4.8 mg/kg and 6.0 mg/kg for treatment, and a study of in vivo pharmacokinetics was conducted in 67 Chinese patients suffering from solid tumors with HER2 expression. Results showed that the compound I-1 exhibited nonlinear pharmacokinetic characteristics, and its half-life period was prolonged with increase in administration dose. The mean half-life period of the groups of 4.8 and 6.0 mg/kg doses were 8.5 and 8.8 days, respectively. In the first cycle of administration, the Cₘₐₓ and AUC of free toxin accounted for 0.1% and 0.2% of the total ADC molar concentration, respectively, indicating that the compound I-1 had a low toxin shedding rate and was stable in blood circulation.

### Example 13: Clinical Safety Study

In the clinical trial safety studies of currently marketed HER2-ADC drugs, both Kadcyla© and Enhertu© drugs would cause a certain proportion of adverse effects, including thrombocytopenia, vomiting, diarrhea, neutropenia, leukopenia, anemia, and nausea (see Dieras V, Miles D, Verma S, et al: Trastuzumab emtansine versus capecitabine plus lapatinib in patients with previously treated HER2-positive advanced breast cancer (EMILIA): a descriptive analysis of final overall survival results from a randomised, open-label, phase 3 trial. Lancet Oncol 18:732-742, 2017; Modi S, Saura C, Yamashita T, et al: Trastuzumab Deruxtecan in Previously Treated HER2-Positive Breast Cancer. N Engl J Med 382:610-621, 2020). The compound I-1 of the present application exhibited low incidence in adverse effects such as thrombocytopenia, vomiting, diarrhea, neutropenia and nausea, and was superior to the marketed Kadcyla© and Enhertu© in clinical safety.

In the clinical safety study of the present application, the patients suffering from HER2-positive breast cancer were treated with the compound I-1 at doses of 4.8 mg/kg and 6.0 mg/kg, once every three weeks at the first day of each cycle of administration. The safety results from current 58 evaluable patients are as follows:

TABLE 1

| | Thrombo- | Neutro- | Leuko- | | | | | Pulmonary |
|---|---|---|---|---|---|---|---|---|
| | cytopenia | penia | penia | Anemia | Nausea | Vomiting | Diarrhea | Toxicity |
| Total | 5.2% | 6.9% | 10.3% | 24.1% | 12.1% | 6.9% | 12.1% | 5.2% |
| Incidence | | | | | | | | |

Moreover, the compound I-1 of the present invention exhibited no adverse effects such as ≥grade 3 thrombocytopenia, neutropenia, nausea, vomiting, diarrhea, and pulmonary toxicity, and a very low proportion of ≥grade 3 anemia and ≥grade 3 leukopenia (the incidences were 3.4% and 1.7%, respectively) in the above clinical safety tests, as shown in Table 2 below:

TABLE 2

| | Thrombo- | Neutro- | Leuko- | | | | | Pulmonary |
|---|---|---|---|---|---|---|---|---|
| | cytopenia | penia | penia | Anemia | Nausea | Vomiting | diarrhea | Toxicity |
| | (≥Grade 3) | (≥Grade 3) | (≥Grade 3) | (≥Grade 3) | (≥Grade 3) | (≥Grade 3) | (≥Grade 3) | (≥Grade 3) |
| Incidence | 0 | 0 | 1.7% | 3.4% | 0 | 0 | 0 | 0 |

In conclusion, the compound I-1 of the present application exhibited, in the clinical safety study, no pulmonary toxicity, and very low incidences and severities of myelosuppression and gastrointestinal toxicity and adverse effects, and had improved safety as compared with the marketed drugs such as Kadcyla® and Enhertu®

1. A method for the prevention and/or treatment of a cancer with HER2 expression, amplification or mutation, comprising administering to a subject in need thereof a therapeutically effective amount of an antibody-drug conjugate of Formula (I), a pharmaceutically acceptable salt, a stereoisomer or a metabolite thereof, or a solvate of the foregoing, 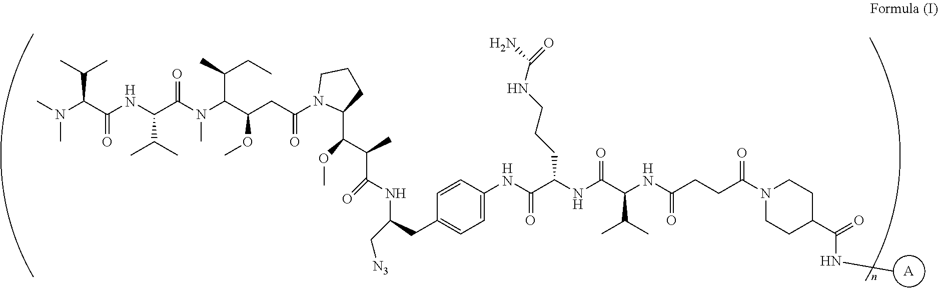
wherein
A is a moiety obtained by the removal of n amino groups from an anti-HER2 antibody or an active fragment or variant thereof, preferably a moiety obtained by the removal of n amino groups from trastuzumab or pertuzumab; and
n is an integer selected from a group consisting of 1-10, e.g., 2-10 or 2-8; preferably, n is an integer of 1, 2, 3, 4, 5, 6, 7 or 8.

2. The method according to claim 1, wherein the cancer is a cancer with HER2 low-expression;
preferably, the cancer with HER2 low-expression is a cancer with an HER2 expression level of IHC 1+ or IHC 2+/FISH negative; and
preferably, the cancer with HER2 low-expression is head and neck cancer, preferably head and neck squamous cell carcinoma with an HER2 expression level of IHC 2+.

3. The method according to claim 1, wherein the cancer is a cancer with HER2 mutation and is selected from a group consisting of breast cancer, biliary tract cancer, ovarian cancer, lung cancer, colorectal cancer, salivary gland cancer, head and neck cancer, endometrial cancer, cervical cancer, liver cancer, and upper gastrointestinal cancer;
preferably, the salivary gland cancer is one or more of parotid gland carcinoma, salivary duct carcinoma, adenoid cystic carcinoma, submaxillary gland carcinoma, sublingual gland carcinoma, lingual gland carcinoma, minor salivary gland carcinoma, labial gland carcinoma, and retromolar gland carcinoma; and
preferably, the upper gastrointestinal cancer is esophageal adenocarcinoma or biliary duct cancer.

4. The method according to claim 12, wherein the cancer is a breast cancer with HER2 expression, or a breast cancer which has been treated ineffectively with at least one anti-HER2 therapeutic regimen in the past;
preferably, the breast cancer with HER2 expression is unresectable breast cancer, and the breast cancer which has been treated ineffectively with at least one anti-HER2 therapeutic regimen in the past is HER2-positive unresectable breast cancer; and
preferably, the unresectable breast cancer is unresectable locally advanced, recurrent or metastatic breast cancer, and the HER2-positive unresectable breast cancer is HER2-positive unresectable locally advanced, recurrent or metastatic breast cancer.

5. The method according to claim 1, wherein
the antibody-drug conjugate of Formula (I), the pharmaceutically acceptable salt, the stereoisomer or the metabolite thereof, or the solvate of the foregoing is administered at a dose of 0.1-15 mg/kg body weight, preferably at a dose of 0.1-10 mg/kg, 0.2-8 mg/kg, and 0.3-6 mg/kg, and more preferably at a dose of 0.3 mg/kg, 1.2 mg/kg, 3.6 mg/kg, 4.8 mg/kg, 6 mg/kg, and 7.2 mg/kg;
preferably, a route of administration is oral administration, parenteral administration, or transdermal administration, and the parenteral administration is selected from a group consisting of intravenous injection, subcutaneous injection, and intramuscular injection; and
preferably, the antibody-drug conjugate of Formula (I), the pharmaceutically acceptable salt, the stereoisomer or the metabolite thereof, or the solvate of the foregoing is in an injectable form, e.g., subcutaneous or intravenous injection; wherein a particularly preferred injectable form is an injection liquid or lyophilized powder for injection comprising the antibody-drug conjugate of Formula (I), the pharmaceutically acceptable salt, the stereoisomer or the metabolite thereof, or the solvate of the foregoing, as well as a buffering agent, a stabilizing agent, a pH regulating agent, and optionally a surfactant; wherein the buffering agent is selected from one or more of acetates, citrates, succinates, and phosphates; the stabilizing agent is selected from a group consisting of saccharides and amino acids, preferably disaccharides, such as sucrose, lactose, mycose, and maltose; the surfactant is selected from a group consisting of polyoxyethylene hydrogenated castor oil, glycerin fatty acid ester, and polyoxyethylenesorbitan fatty acid ester, and preferably the polyoxyethylenesorbitan fatty acid ester is polysorbate 20, 40, 60, or 80, most preferably polysorbate 20; and the pH regulating agent is selected from one or more of sodium hydroxide, lithium hydroxide, and potassium hydroxide.

6. The method according to claim 1, wherein the antibody-drug conjugate of Formula (I) has a structure represented by Formula (I-1), 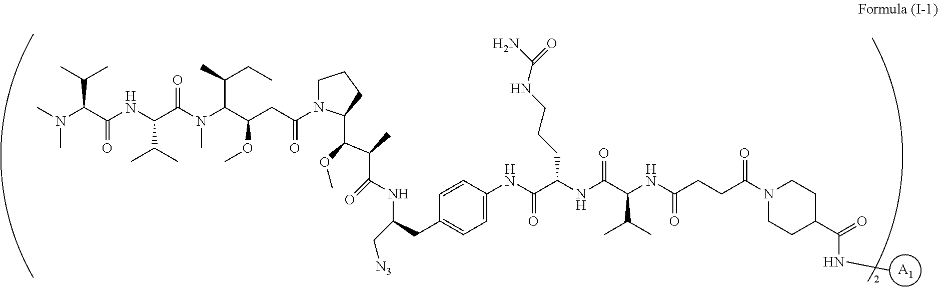
wherein A1 is a moiety obtained by the removal of 2 amino groups from trastuzumab.

7. The method according to claim 1, wherein after the patient is treated with the antibody-drug conjugate of Formula (I), the pharmaceutically acceptable salt, the stereoisomer or the metabolite thereof, or the solvate of the foregoing, an incidence of thrombocytopenia in the patient is 10% or lower, preferably 8% or lower, and more preferably 5% or lower; and an incidence of thrombocytopenia of grade 11 and above is 5% or lower, preferably 3% or lower, more preferably 1% or lower, and most preferably 0;
and/or
after the patient is treated with the antibody-drug conjugate of Formula (I), the pharmaceutically acceptable salt, the stereoisomer or the metabolite thereof, or the solvate of the foregoing, an incidence of neutropenia in the patient is 20% or lower, preferably 10% or lower, more preferably 8% or lower, and further preferably 5% or lower; and an incidence of neutropenia of grade 11 and above is 5% or lower, preferably 3% or lower, more preferably 1% or lower, and most preferably 0;
and/or
after the patient is treated with the antibody-drug conjugate of Formula (I), the pharmaceutically acceptable salt, the stereoisomer or the metabolite thereof, or the solvate of the foregoing, an incidence of leukopenia in the patient is 10% or lower, preferably 8% or lower, more preferably 5% or lower, and further preferably 3% or lower; and an incidence of leukopenia of grade 11 and above is 5% or lower, preferably 3% or lower, more preferably 1% or lower, and most preferably 0;
and/or
after the patient is treated with the antibody-drug conjugate of Formula (I), the pharmaceutically acceptable salt, the stereoisomer or the metabolite thereof, or the solvate of the foregoing, an incidence of anemia in the patient is 30% or lower, preferably 25% or lower, and more preferably 20% or lower; and an incidence of anemia of grade 11 and above is 10% or lower, preferably 8% or lower, more preferably 5% or lower, and further preferably 3% or lower;
and/or
after the patient is treated with the antibody-drug conjugate of Formula (I), the pharmaceutically acceptable salt, the stereoisomer or the metabolite thereof, or the solvate of the foregoing, an incidence of nausea in the patient is 30% or lower, preferably 25% or lower, more preferably 20% or lower, and further preferably 15% or lower; and an incidence of nausea of grade 11 and above is 5% or lower, preferably 3% or lower, more preferably 1% or lower, and most preferably 0;
and/or
after the patient is treated with the antibody-drug conjugate of Formula (I), the pharmaceutically acceptable salt, the stereoisomer or the metabolite thereof, or the solvate of the foregoing, an incidence of vomiting in the patient is 30% or lower, preferably 20% or lower, more preferably 15% or lower, and further preferably 10% or lower; and an incidence of vomiting of grade 11 and above is 5% or lower, preferably 3% or lower, more preferably 1% or lower, and most preferably 0;
and/or
after the patient is treated with the antibody-drug conjugate of Formula (I), the pharmaceutically acceptable salt, the stereoisomer or the metabolite thereof, or the solvate of the foregoing, an incidence of diarrhea in the patient is 30% or lower, preferably 20% or lower, more preferably 15% or lower, further preferably 10% or lower, and still further preferably 8% or lower; and an incidence of diarrhea of grade 11 and above is 5% or lower, preferably 3% or lower, more preferably 1% or lower, and most preferably 0;
and/or
after the patient is treated with the antibody-drug conjugate of Formula (I), the pharmaceutically acceptable salt, the stereoisomer or the metabolite thereof, or the solvate of the foregoing, an incidence of pulmonary toxicity in the patient is 5% or lower, preferably 3% or lower, more preferably 1% or lower, and most preferably 0; and an incidence of pulmonary toxicity of grade 11 and above is 5% or lower, preferably 3% or lower, more preferably 1% or lower, and most preferably 0.

8. The method according to claim 2, wherein the cancer with HER2 low-expression is selected from a group consisting of salivary gland cancer, breast cancer, gastric cancer, gastroesophageal junction cancer, esophageal cancer, lung cancer, colorectal cancer, urothelial cancer, biliary tract cancer, head and neck cancer, ovarian cancer, endometrial cancer, cervical cancer, pancreatic cancer, and liver cancer,
preferably, the esophageal cancer is esophageal adenocarcinoma or esophageal squamous cell carcinoma.

9. The method according to claim 2, wherein the cancer with HER2 low-expression is salivary gland cancer.

10. The method according to claim 9, wherein the salivary gland cancer is parotid gland carcinoma with an HER2 expression level of IHC 2+ or salivary duct carcinoma with an HER2 expression level of IHC 1+.

11. The method according to claim 2, wherein the cancer with HER2 low-expression is breast cancer, preferably breast cancer with an HER2 expression level of IHC 1+.

12. The method according to claim 2, wherein the salivary gland cancer is one or more of parotid gland carcinoma, salivary duct carcinoma, adenoid cystic carcinoma, submaxillary gland carcinoma, sublingual gland carcinoma, lingual gland carcinoma, minor salivary gland carcinoma, labial gland carcinoma, and retromolar gland carcinoma.

13. The method according to claim 2, wherein the cancer is a cancer with HER2 over-expression;
preferably, the cancer with HER2 over-expression is a cancer with an HER2 expression level of IHC 2+/FISH positive or IHC 3+;
preferably, the cancer with HER2 over-expression is selected from a group consisting of breast cancer, gastric cancer, gastroesophageal junction cancer, esophageal cancer, lung cancer, colorectal cancer, urothelial cancer, salivary gland cancer, biliary tract cancer, head and neck cancer, ovarian cancer, endometrial cancer, cervical cancer, pancreatic cancer, liver cancer, gallbladder cancer, and bladder cancer;
preferably, the cancer with HER2 over-expression is selected from a group consisting of breast cancer, bladder cancer, colorectal cancer, gallbladder cancer, salivary gland cancer, and head and neck cancer, preferably breast cancer, bladder cancer, colorectal cancer, gallbladder cancer, parotid gland carcinoma or head and neck squamous cell carcinoma with an HER2 expression level of IHC 3+; and
preferably, the esophageal cancer is esophageal adenocarcinoma or esophageal squamous cell carcinoma.

14. The method according to claim 13, wherein the salivary gland cancer is one or more of parotid gland carcinoma, salivary duct carcinoma, adenoid cystic carcinoma, submaxillary gland carcinoma, sublingual gland carcinoma, lingual gland carcinoma, minor salivary gland carcinoma, labial gland carcinoma, and retromolar gland carcinoma.

15. The method according to claim 1, wherein the cancer is liver cancer, and preferably the liver cancer is intrahepatic cholangiocarcinoma or hepatocellular carcinoma.

16. The method according to claim 1, wherein the cancer with HER2 amplification is selected from a group consisting of breast cancer, gastroesophageal junction adenocarcinoma, ovarian cancer, colorectal cancer, lung cancer, gallbladder cancer, esophageal cancer, and upper gastrointestinal cancer;
preferably, the esophageal cancer is esophageal adenocarcinoma or esophageal squamous cell carcinoma; and
preferably, the upper gastrointestinal cancer is esophageal adenocarcinoma or biliary duct cancer.

17. The method according to claim 4, wherein the breast cancer is unresectable locally advanced, recurrent or metastatic breast cancer with HER2 expression, or HER2-positive unresectable locally advanced, recurrent or metastatic breast cancer which has been treated ineffectively with at least one anti-HER2 therapeutic regimen in the past.

18. The method according to claim 4, wherein the at least one anti-HER2 therapeutic regimen is selected from a group consisting of: (1) trastuzumab and/or pertuzumab combined chemotherapy; (2) tucatinib; (3) neratinib; (4) T-DM1; (5) pyrotinib combined chemotherapy; (6) lapatinib combined chemotherapy; and (7) tucatinib or pyrotinib or lapatinib+trastuzumab+chemotherapy, wherein the trastuzumab and/or pertuzumab are/is replaceable with biosimilars thereof.

19. The method according to claim 5, wherein the antibody-drug conjugate of Formula (I), the pharmaceutically acceptable salt, the stereoisomer or the metabolite thereof, or the solvate of the foregoing is administered at a frequency of once a day, twice a day, three times a day, once a week, once every two weeks, once every three weeks, once every four weeks or once a month, once every five weeks, or once every six weeks.

20. The method according to claim 1, wherein the patient has also received a pretreatment step prior to the prevention and/or treatment;
preferably, the pretreatment is performed using an antipyretic analgesic or an antihistamine drug;
preferably, the antipyretic analgesic is selected from a group consisting of acetaminophen and ibuprofen;
preferably, the antihistamine drug is selected from a group consisting of diphenhydramine and promethazine;
preferably, the patient has also received other treatments selected from a group consisting of a surgical therapy, a radiotherapy, and a drug therapy;
preferably, the drug therapy is selected from a group consisting of an anti-HER2 therapy, a hormone therapy, and a chemotherapy;
preferably, the anti-HER2 therapy is selected from a group consisting of an anti-HER2 antibody drug, such as a monoclonal antibody, an antibody-drug conjugate (ADC), or a bispecific antibody, or a chemical drug targeting HER2, such as lapatinib, neratinib, afatinib, or varlitinib; preferably, the drug targeting HER2 is trastuzumab or pertuzumab, or a biosimilar thereof, such as ABP980, GB221, MYL-1401O, CT-P6, EG12014, HD201, ONS-1050, PF-05280014, Ontruzant or HLX02, or is an antibody-drug conjugate with trastuzumab or pertuzumab or a biosimilar thereof as a targeting component, such as an antibody-cytotoxic drug conjugate obtained by conjugation of the targeting component to DM1, DM4, MMAE, or MMAF, e.g., T-DM1;
preferably, the hormone is selected from a group consisting of estrogen receptor (ER) blockers such as tamoxifen, toremifene, fulvestrant, letrozole and anastrozole, ER modulators, or aromatase inhibitors; and
preferably, the chemotherapy drug is selected from a group consisting of paclitaxel, paclitaxel-albumin, docetaxel, gemcitabine, capecitabine, tegafur, carboplatin, vinorelbine, cyclophosphamide, and pharmorubicin.
